(19) United States
(12) Plant Patent Application Publication
(10) Pub. No.: US 2026/0283061 P1
(43) Pub. Date: Sep. 17, 2026

(54) Dicentra plant named '17001GR1'

(50) ;

(71) Applicant: A. Blom Holding B.V., Vleuten (NL)

(72) Inventor: Arie Blom, Vleuten (NL)

(73) Assignee: A. Blom Holding B.V., Vleuten (NL)

(21) Appl. No.: 19/554,454

(22) Filed: Mar. 2, 2026

(30) Foreign Application Priority Data
Mar. 12, 2025 (QZ) 2025/0456

(51) Int. Cl. A01H 6/64; A01H 5/02

(52) U.S. Cl. CPC A01H 6/64; A01H 5/02
USPC PLT/426

(57) ABSTRACT

A new and distinct cultivar of Dicentra plant named ‘17001GR1’ is disclosed, characterized by bright green foliage with red flowers. Plants are large, robust, and vigorous. The new cultivar is a Dicentra, typically used for ornamental horticulture purposes.

Latin name of the genus and species: Dicentra spectabilis.

Cultivar denomination:

‘17001GR1’.

### BACKGROUND OF THE INVENTION

The new cultivar is the product of a planned breeding program by the inventor, to develop new Dicentra cultivars with larger, more robust plant habits. The seed parent is an internal Dicentra selection known as ‘15-002groen’. The self-pollination resulting in the new cultivar was made in May 2017 at a nursery in Boskoop, the Netherlands. discovered and selected by the inventor in July 2020, at the same nursery in Boskoop, the Netherlands.

Asexual reproduction of the new cultivar ‘17001GR1’ by tissue culture was performed at a nursery in Hoogkarspel, the Netherlands in July 2020, and has shown that the unique features of this cultivar are stable and reproduced true to type in successive generations.

### SUMMARY OF THE INVENTION

The cultivar ‘17001GR1’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of ‘17001GR1’ These characteristics in combination distinguish ‘17001GR1’ as a new and distinct Dicentra cultivar:
1. Bright green foliage with red flowers.
2. Very suitable for large pot sizes.
3. Vigorous growth habit.
4. Easy to propagate and grow.
5. Good root system.

### PARENT COMPARISON

Plants of the new cultivar ‘17001GR1’ are similar to plants of the seed parent in most horticultural characteristics. However, plants of the new cultivar ‘17001GR1’ differ in the following:
1. Flower color of the new cultivar is red, while the flower color of the seed parent is pink.
2. Branch color of the new cultivar is more red than the branch color of the seed parent.

### COMMERCIAL COMPARISONS

Plants of the new cultivar ‘17001GR1’ are similar to plants of the commercial cultivar, Dicentra ‘Valentine’, unpatented, in most horticultural characteristics, however, plants of the new cultivar ‘17001GR1’ differ in the following ways:
1. Plants of the new cultivar are larger, more robust and vigorous than plants of this comparator.

Plants of the new cultivar ‘17001GR1’ are similar to plants of the commercial cultivar, Dicentra ‘Alba’, unpatented, in most horticultural characteristics, however, plants of the new cultivar ‘17001GR1’ differ in the following ways:
1. Foliage color of the new cultivar is darker green than the foliage color of this comparator.
2. Flower color of the new cultivar is red, while the flower color of this comparator is white.
3. Plants of the new cultivar produce more branches than plants of this comparator.

### BRIEF DESCRIPTION OF THE PHOTOGRAPHS

The photographs were taken using conventional techniques and equipment. While the colors in these photographs may display variances of color as compared to the living cultivar, due to LRV (light reflectance value), they are as accurate as possible using conventional photographic techniques. Colors in the photographs may appear to differ slightly from the color values cited in the botanical description, which accurately describe the colors of new Dicentra ‘17001GR1’.

FIG. 1 illustrates in full color a plant of Dicentra ‘17001GR1’, grown outdoors in Boskoop, the Netherlands. This plant is approximately 1 year old.

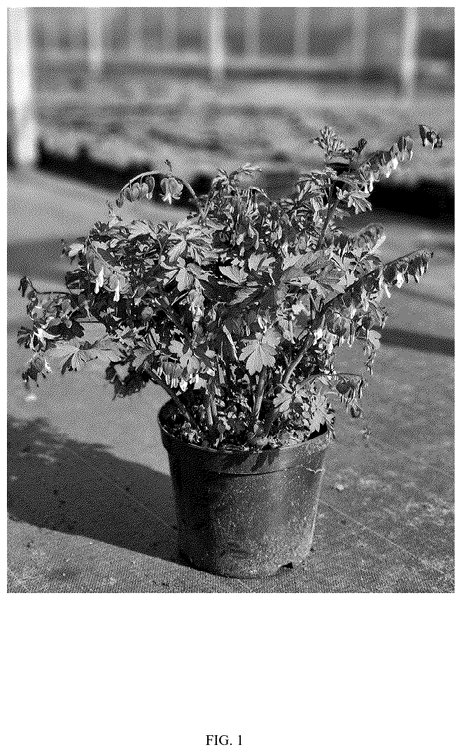

FIG. 2 illustrates in full color a close-up view of flowers of Dicentra ‘17001GR1’, from the plant in FIG. 1.

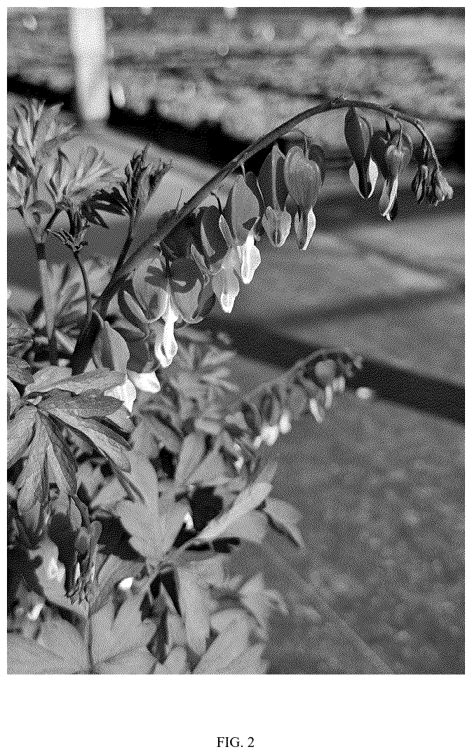

### DETAILED BOTANICAL DESCRIPTION

In the following description, color references are made to The Royal Horticultural Society Color Chart 2015, except where general terms of ordinary dictionary significance are used. The following observations and measurements describe ‘17001GR1’ plants grown at a commercial nursery. The growing temperature ranged from approximately 12° C. to 24° C. during the day, and 20° C. to 12° C. at night. No artificial light, photoperiodic treatments or chemical treatments were given to the plants. Natural light conditions were approximately 9500 foot candles of light.

Measurements and numerical values represent averages of typical plant types.
Botanical classification: Dicentra spectabilis ‘17001GR1.’
Propagation:
   Type of propagation typically used.—Tissue culture.
Plant:
   Age of plant described.—Approximately 18 months.
   Container size of the plant described.—23 cm.
   General plant description.—Herbaceous perennial.
   Overall plant shape.—Flattened globular.
   Growth habit.—Broad spreading-upright.
   Plant spread.—Approximately 55.6 cm.
   Plant height.—Approximately 43.4 cm to top of foliar plane; 36.5 cm to top of floral plane.
   Growth rate.—Moderate.
   Plant vigor.—Moderate to high.
   Branching habit.—Main stem grows from the base, no lateral branches.
   Pinching.—Not required.
   Number of primary (main) branches per plant.—Average 8.
   Number of secondary (lateral) branches per plant.—None.
   Branches.—Length: Average 28.9 cm. Diameter: Average 0.75 cm. Internode length: Average 6.6 cm. Appearance and shape: Rounded, slightly angled. Luster: Moderately glossy, but covered with a very thin, waxy layer, colored RHS Greyed-Green 194A. Aspect/angle from vertical: Average angle 67.5°. Strength: Moderately strong. Color, developing: RHS Yellow-Green 147C. Color, mature: RHS Yellow-Green 148A, slightly tinged Grey-Brown 199A and 199B. Color at internodes: RHS Yellow-Green 148A, slightly tinged Grey-Brown 199A and 199B. Pubescence: None.
Foliage:
   Leaf.—Leaf arrangement: Alternate. Compound or single: Compound. Number of leaves per branch: Average 5. Number of leaflets per leaf: Average 3. Whole leaf shape: Broad ovate. Whole leaf aspect: Slightly carinate. Whole leaf length: Average 16.4 cm. Whole leaf width: Average 15.7 cm. Leaflet shape: Trifoliolate. Leaflet length: Average 9.5 cm. Leaflet width: Average 8.9 cm. Leaf/leaflet apex: Acute. Leaf/leaflet base: Attenuate. Leaf/leaflet margin: Entire, lobed to cleft. Leaf/leaflet lobes: Lobed to cleft. Leaf/leaflet texture, upper side: Smooth, glabrous. Leaf/leaflet texture, under side: Smooth, glabrous. Leaf/leaflet pubescence: None. Leaf/leaflet rugosity: Non-rugose. Leaf/leaflet luster, upper side: Matte. Leaf/leaflet luster, under side: Matte. Leaf/leaflet color, young, upper side: RHS Yellow-Green 146A, slightly tinged and veined Greyed-Purple 183B. Leaf/leaflet color, young, under side: RHS Yellow-Green 148A, slightly tinged and veined Greyed-Purple 183B. Leaf color, mature, upper side: RHS Green 137A, tips tinged Greyed-Purple 183A. Leaf color, mature, under side: RHS Greyed-Green 191A, tips and margins tinged Greyed-Red 178A and 178B. Venation pattern: Pinnate. Mature leaf venation color, upper side: RHS Greyed-Red 178C. Mature leaf venation color, under side: RHS Yellow-Green 148A.
   Petiole.—Petiole length: Average 9.5 cm. Petiole diameter: 0.35 cm. Petiole texture: Smooth, glabrous. Petiole luster: Slightly glossy. Petiole strength: Moderately strong. Petiole color, upper side: RHS Grey-Brown N199A, main vein Greyed-Orange 174B and 174C. Petiole color, under side: RHS Grey-Brown 199A.
   Rachis.—Average length: 8.1 cm. Average diameter: 0.2 cm. Texture: Both sides smooth and glabrous and slightly glossy. Color: Upper side: RHS Yellow-Green 152A, main vein 153A and 153B. Under side: RHS Yellow-Green 152C to Greyed-Green 199B.
Flower:
   Inflorescence.—Arrangement: Axillary and terminal racemes. Type and form: Raceme. Height: Average 14 cm (including peduncle). Diameter: Average 3.5 cm. Quantity of flowers per inflorescence: Average 13. Quantity of flowers per inflorescence: Average 75. Quantity of flower buds per plant: Average 25. Quantity of flowers and flower buds per plant: Average 100. Natural flowering season: April to late May (in the Netherlands). Time to flower or response time: Average 10 months.
   Bud.—Shape: Lower half heart-shaped, upper half obovate, with two wings. Length: Average 1.8 cm. Diameter: Average 0.8 cm. Texture: Glabrous. Luster: Matte. Color: Lower half: RHS Greyed-Red 179A. Upper half: RHS Greyed-Orange 173A and 173B, winged White N155A.
   Flower.—Type and form: Irregular, typical for Dicentra, single. Aspect: Drooping. Flowering habit: Moderate. Fragrance: None. Shape: Irregular, typical for Dicentra, consisting of 2 pairs of petals, 2 anthers and a single pistil. Height: Average 3.1 cm. Diameter: Flowers flattened, average diameter at widest: 2.8 cm, average diameter at narrowest: 0.9 cm. Longevity: Average 10 days on plant. Persistence: Self-cleaning.
   Petals.—Arrangement: Cruciform. Number: Average 4. Whorls: Average 2. Length: Outer petals: Average 1.9 cm. Inner petals: Average 2.5 cm. Width: Outer petals: Flattened, average width at widest: 1.5 cm, average width at narrowest: 0.9 cm. Inner petals: Average 0.5 cm. Shape: Outer petals: Folded, ovate. Inner petals, lower: Oblong. 8Inner petals, upper: Obovate, winged. 9 Tip: Outer and inner petals acute. Base: Outer petals: Broad cuneate. Inner petals: Cuneate. Margin: Entire. Texture: Both sides smooth, glabrous. Rugosity: Both sides non-rugose. Luster: Both sides matte. Color: When opening, upper side: Outer petal: RHS Greyed-Red 181C and 181D. Inner petal: Lower half RHS White N155A, tinged Greyed-Purple 185D in the center; upper half White 155C, margined Yellow-Green 144C; top Greyed-Purple N186C, margined White NN155B. When opening, under side: Outer petal: RHS Greyed-Red 180A and 180B, tipped Greyed-Purple 183A. Inner petal: Lower half RHS White N155A, fading to Greyed-Purple 185D towards base; upper half Yellow-Green N144D, winged transparent White N155A; top Greyed-Green 197C and 197D; margined White 155C. Fully opened, upper side: Outer petal: RHS Greyed-Red 185C and 185D. Inner petal: Lower half RHS White N155A, tinged Greyed-Purple 186C in the center; upper half White NN155A, margined Yellow 2B; top Greyed-Purple N186C, margined White NN155B. Fully opened, under side: Outer petal: RHS Greyed-Red 182A and 182B, tipped Yellow-Green 145D. Inner petal: Lower half RHS White N155A, fading to Greyed-Purple 185D towards base; upper half Yellow 1C, winged transparent White N155A; top Greyed-Purple 183B and 183C, margined White 155C. Fading, upper side: Outer petal: RHS Greyed-Purple 186B and 186C. Inner petal: Lower half RHS White N155A, tinged Greyed-Purple 186D in the center; upper half White NN155A, margined Yellow 2A; top Greyed-Purple N186C, margined White NN155B. Fading, under side: Outer petal: RHS Greyed-Purple 185C, tipped N187A. Inner petal: Lower half RHS White N155A, fading to Greyed-Purple 186D towards base; upper half Yellow 2B; winged transparent White N155A; top Greyed-Purple183B and 183C; margined White 155C.
   Calyx.—No sepals present, calyx consisting of petals only. Shape: Flattened. Length: Average 3.1 cm. Diameter: Flowers flattened, average diameter at widest: 2.8 cm, average diameter at narrowest: 0.9 cm.
   Peduncle.—Length: Average 13.5 cm. Diameter: Average 0.3 cm. Angle: Average 15°. Strength: Moderately strong. Texture: Smooth, glabrous. Luster: Slightly glossy. Color: RHS Yellow-Green 146A, upper side strongly tinged Greyed-Orange 176A.
   Pedicel.—Length: Average 1.1 cm. Diameter: Average 0.1 cm. Angle: Average 80° to peduncles. Strength: Moderately strong. Texture: Smooth, glabrous. Luster: Slightly glossy. Color: RHS Greyed-Orange 176A and 178B.
   Floral bracts.—Number: A single bract at the base of each pedicel. Shape: Narrow linear. Average length: 0.4 cm. Average width: 0.5 mm. Tip: Narrow acute. Base: Cuneate. Margin: Entire. Texture: Smooth, glabrous. Color: RHS Greyed-Orange 178B and 178C.
Reproductive organs:
   Stamens.—Number: Stamens arranged in 2 bundles, each bundle has a single filament, splitting in 2 or 3 parts at the apex. Filament length: Average 2.1 cm. Filament color: Lower two-thirds RHS White N155A, tinged Greyed-Purple 185C; upper one-third White NN155C.
   Anther.—Shape: Basifixed, elliptic. Length: Average 0.1 cm. Width: Average 0.5 mm. Color: RHS Yellow 11A. Pollen amount: Moderate. Pollen color: RHS Yellow 12B. Pistil number: Average 1.Pistil length: Average 0.9 cm. Stigma shape: Club-shaped. Stigma length: Average 0.1 cm. Stigma diameter: Average 0.1 cm. Stigma color: RHS Yellow-Green 153D. Style length: Average 0.8 cm. Style color: RHS Yellow-Green 145A. Ovary color: RHS Yellow-Green 143C.
Other characteristics:
   Fruit and seed.—Not observed to date.
   Disease/pest resistance.—Similar to other Dicentra cultivars.
   High temperature tolerance.—Unknown, at least tolerant to temperatures up to 35° C.
   Low temperature tolerance.—Unknown, at least hardy to USDA zone 4 (−28° C.).
   USDA hardiness zones.—USDA zones 4 to 9.
   Tolerance to rain.—Moderate.
   Tolerance to wind.—Moderate.

What is claimed is:

1. A new and distinct cultivar of Dicentra plant named ‘17001GR1’ as herein illustrated and described.
